(19) United States
(12) Patent Application Publication
(10) Pub. No.: US 2010/0195093 A1
(43) Pub. Date: Aug. 5, 2010

(54) CHARACTERIZATION OF OPTICAL SYSTEMS

(75) Inventors: Arthur Ho, Coogee (AU); Klaus Ehrmann, Coogee (AU)

Correspondence Address: MERCHANT & GOULD PC, P.O. BOX 2903, MINNEAPOLIS, MN, 55402-0903, US

(73) Assignee: The Institute for Eye Research Limited, Sydney (AU)

(21) Appl. No.: 12/527,381

(22) PCT Filed: Feb. 14, 2008
(86) PCT No.: PCT/AU08/00183

§ 371 Date: Apr. 15, 2010

(30) Foreign Application Priority Data
Feb. 14, 2007 (AU) 2007900710

(51) Int. Cl. G01M 11/02; A61B 3/10

(52) U.S. Cl. 356/124; 351/221; 351/246

(57) ABSTRACT

An instrument and method is for characterizing the optical properties of an optical system, such as a lens, another optical device or the human eye, over an optical surface of the optical system. In one example, an incident beam is scanned over the surface of a lens to generate an emergent beam that is divided by a beam-splitter into two portions that are directed to respective two-dimensional detector arrays located at different optical distances from the lens. The detector arrays output the lateral coordinates of the points of incidence of the respective emergent beam portions so that the angle of emergent beam with respect to the optical axis or incident beam can be accurately determined. Determining the variation in the angle of the emergent beam over the surface of the lens allows many important optical characteristics of the lens to be characterized and mapped onto to the surface of the lens.

### BACKGROUND OF THE INVENTION

1. Technical Field

This invention broadly relates to methods, instruments and apparatus for use in the characterization of optical systems, devices and elements. It is applicable to the determination of reflective and refractive characteristics of figured optical elements such as lenses, mirrors and even complex optical systems such as natural or model eyes and optical instruments.

One use of such methods and apparatus is mapping or spatially resolving refractive power over the area of a lens, which is sometimes referred to as the determination of wave-front aberrations of the lens. The instruments or apparatus concerned include wave-front sensors that can be used with the human eye, with isolated lenses, sets of lenses, mirrors and figured reflective or refractive surfaces (collectively referred to herein as ‘optical system(s)’). Of particular practical interest is mapping the refractive power of ophthalmic lenses, and more specifically of soft contact lenses for the purpose of quality control in production, experimental use and prescription.

The reflective and refractive characteristics of interest are data-sets here—referred to as ‘power maps’ indicating the variation of sphere, prism, cylinder and/or axis components, Zernike descriptors for higher and lower order aberrations, optical mean transfer functions, averaged power profiles, or the like, over an optical surface of the optical system of interest. Visualization of the variation of selected characteristics over an optical surface provides a valuable way of checking the performance of an optical system. This allows efficient assessment of the various refractive, blending and peripheral zones of soft contact lenses, for example. The methods and apparatus of the present invention may also be applied to mapping the optical characteristics of the intact human eye onto—say—the surface of the cornea by making use of light reflected or scattered from the retina or another surface of the eye.

For convenience, therefore, such data-sets and their generation will be herein referred to as ‘power maps’ and ‘power mapping’ without intending to suggest that either visualization is essential or that simple refractive power is the only characteristic of interest.

2. Description of Related Art

A variety of wave-front sensing methods are used for assessment of optical aberrations of lenses. An overview of these methods has been given by J. M. Geary. ‘Introduction to Wave-front Sensors’ SPIE 1995, ISBN-13: 978-0819417015. Hartmann-Shack and ray-tracing techniques are commonly used for mapping the optical aberrations of lenses and are suited to mapping the complex optical characteristics of the natural eye. These techniques were explained and compared by E. Moreno-Barriuso and R. Navarro in a paper entitled, ‘Laser Ray Tracing versus Hartmann-Shack sensor for measuring optical aberrations in the human eye’ [J. Opt. Soc. Am. A/Vol. 17, No. 6/June 2000].

In the Hartmann-Shack technique, a plate consisting of a uniform array of micro-lenslets is positioned in the optical path after the lens under test so that a corresponding array of image spots is projected onto the objective plane, the departure of the image array from uniformity indicating aberration (or local variation in optical power). In this technique, it is not necessary to know the precise distance of an optical surface or axis from the instrument or target for good results.

In ray-tracing, a narrow laser beam is scanned over the surface of the lens to build up a similar image array, one spot at a time. While ray-tracing allows a more finely detailed power map to be generated because spot sizes can be less than for Hartmann-Shack, the Hartmann-Shack technique allows rapid assessment since all rays/spots are processed at once rather than serially. It should also be noted that ray-tracing methods assume that the position of a lens surface along the optical axis of the instrument is known with precision, which is problematic in the case of optical devices with small, strongly curved or figured surfaces, or which are flexible like soft contact lenses. Wave-front aberrations are commonly described by means of Zernike Polynomials. These polynomials can mathematically be converted into refractive power maps to visualize the lower order aberrations within the plain view of the optical device.

An example of a ray-tracing technique for measuring refractive aberration in the human eye is disclosed by U.S. Pat. No. 6,932,475 to Molebny et al [and by Molebny et al in “Principles of Ray Tracing Aberrometry” J. Refractive Surgery, Vol 6 S572-S575 (2000). See also U.S. Pat. Nos. 7,311,400, 7,303,281 and 6,409,345 to Molebny]. In U.S. Pat. No. 6,932,475, for example, an image of a spot reflected from the retina is directed to two linear CCD detectors arranged at the same effective distance from the retina in such a way that the x-coordinate of the spot is recorded by one detector and the y-coordinate of the same spot is simultaneously recorded by the other. In this way, an aberration map can be built by showing the manner in which spots are shifted with respect to the interrogating beam.

Campbell and Hughes [Vision Res. Vol 21 pp 1229-1148, 1981], Glasser and Campbell [Vision Res. Vol 38 No. 2 pp 209-229, 1988] and Roorda and Glasser [Journal of Vision (2004) 4, 250-261] disclosed methods of measuring wave aberrations in isolated animal eye lenses in which the trajectories of an array of narrow incident and refracted laser beams are photographed laterally or from the side. In these methods, a mounted lens is located in a tank of a milky solution that makes visible the incident and emergent rays. The incident rays can be scanned laser beams or a bundle of incident beams directed parallel to the optical axis of the lens under test. The number of rays that can be employed is strictly limited by the need to visualize and distinguish them in side view. However, the slope of each ray was estimated (if crudely) and a contour map of wave-front aberration was constructed. Though not disclosed, it is noted that the refractive power at a spot on the lens can be estimated from the slope of the beam emerging from that spot and, therefore, it should be possible to construct a power map of the lens. The accuracy and resolution of such a map would leave a lot to be desired and the crudeness and laboriousness of the technique makes it quite impractical as a method for generating useful power maps of contact lenses for production quality control. The technique cannot, of course, be applied to eyes in vivo.

Chase et al [Chase R, Keleti S, Norman B R, A Scanning Hartmann Instrument. Proceedings of SPIE Volume 1618, Large Optics II (1991) pp 89-96] presented a method for slope determination of large mirrors whereby a laser beam is scanned across the mirror in a pivoting motion and the reflected beam is captured with a photodetector mounted on an x-y stage. From the lateral position of the detected reflected beam, the slope of the mirror can be determined for each raster spot. The position detection unit can move only in the lateral x-y plane and its axial location is in close proximity to the incoming beam steering scanner to achieve optimum resolution and measurement range.

A concept of using spot detection at two axial planes has been disclosed in U.S. Pat. No. 6,406,146 B1. The apparatus is essentially a Hartmann-Shack ocular wave-front sensor with a beam splitter added after the lenslet array so that a second photodetector can be added at a different axial position to the first one. This second detector helps to extend the measurement range by reducing the ambiguity of overlapping spots, which is the limiting factor with single detector Hartmann-Shack systems.

### BRIEF SUMMARY OF THE INVENTION

From one aspect, this invention involves directing an incident beam onto spots on an optical surface so as to generate an emergent beam for each spot, determining the lateral location of each emergent beam at first and second optical distances from the surface and deriving the optical power at each spot therefrom. Normally this will involve calculating the emergent angle of the emergent beam at each spot. The resulting data can then be used to determine the optical characteristics of the system. Normally, the scanning of the incident beam, the computation of emergent beam angles, the generation of the data-set and its visual presentation will be computer-controlled or mediated. This will allow a wide variety of optical characteristics of the optical system to be generated and, if desired, to be visually mapped onto a representation of the optical surface.

The lateral locations of the emergent beam are preferably determined by employing detector means that includes at least one photodetector array that can be arranged to intercepting at least a portion of the emergent beam at each optical distance and to output the lateral spatial coordinates of the beam at each distance to processor means. The lateral coordinates of an emergent beam at two distances will generally be sufficient to allow the angle of emergent beam to be determined with sufficient precision and, when related to the corresponding incident angle of the beam, the refractive power of the optical system at each spot to be readily computed. The set of such measurements and/or computations over the optical surface then provides a data-set from which as noted above many important optical characteristics of the optical system can be derived and, if desired, displayed or mapped onto the surface.

It is convenient but not essential to ensure that each incident beam is parallel with is a fixed axis of the optical system. Normally, this will be the central optical axis of the system but the fixed axis can be arbitrarily designated. If all incident beams are parallel with one another—and preferably parallel with the optical axis of the system—then variation of the angles of the emergent beams can be used as a proxy for power variation of the optical system over the designated optical surface. If the angle of the incident beam varies from spot to spot, as would be the case where the incident beams are conically scanned from a common point source, then it is necessary to record or calculate the angle of each incident beam with respect to a common datum such as the optical axis and to employ both the incident and the emergent angles for each spot to calculate the ‘spot power’.

It is convenient to use two-dimensional photodetector arrays (for example CCDs or CMOS detectors of the types commonly used in digital cameras) to detect and output, determine and/or derive the intersection coordinates of the emergent beam at each optical distance or plane so that the angle and/or position of the emergent beam can be determined. Various arrangements are envisaged. For example, a single detector array can be moved from one plane to another and beam coordinates can be derived at each location. A single fixed array can be used at one location with one or more beam-dividers that direct portions of the emergent beam to an array via different optical distances; the various portions of the emergent beam being distinguished from one another by encoding, time-division or other forms of multiplexing. Multiple photodetector arrays can be used in-line if selected arrays are moved into or out of the beam path to intercept the emergent beam at a desired distance. Alternatively, it may be possible to obtain photodetector arrays that are sufficiently transparent to allow one to be fixedly located in-line behind the other. Multiple fixed arrays that are not in-line can be employed by using beam-dividers to direct portions of the same emergent beam to each.

Any convenient beam-divider known in the art may be employed, such as partially silvered mirrors, dichroic or polarizing or non-polarizing cubic or pelliclular beam-splitters, rotating or oscillating mirrors, cubes, prisms that act as beam switchers, or beam multiplexers that make use of encoding, variation of optical properties or time-division multiplexing.

While the detector arrays are preferably of the planar two-dimensional ‘area’ type so that each can immediately output the lateral coordinates of the emergent beam at its location, narrow linear detector arrays can also be employed if intersections along specific meridians only are of interest. Otherwise, such linear detector arrays can be rotated or crossed at a location to effectively act as full or partial area arrays.

It will be appreciated from the above that the detector means embraces both the detector arrays and any beam-divider.

In one application, the method may include the step of supporting a hydrated soft contact lens horizontally and directing the incident light beam vertically downward through the lens and dividing the emergent beam at a location below the lens to direct different portions of the refracted emergent beam to respective fixed detectors arranged at different optical distances from the lens. In this case, the contact lens constitutes the optical system and, conveniently, its upper surface constitutes the optical surface over which optical characteristics are to be mapped. As some contact lenses do not have circular peripheral boundaries, have multiple optical zones, are designed to be used in a particular orientation, and/or have minute orientation markings, it is desirable for the incident beam to be scanned over the entire surface of the lens, beyond its peripheral boundary and for the computer software to identify and reproduce the orientation mark.

The use of more than two detectors at different distances can enhance the precision with—or the range over—which the coordinates of the emergent beam can be determined at a given plane or location. The additional detector or detectors can be positioned intercept emergent beams that are deflected more or less normal. For example, when a more powerful lens than normal is being mapped, the emergent beam may be ‘super-deflected’ to such a degree that it misses the more remote of two ‘standard’ detectors. A third detector could thus be positioned to intercept the super-deflected emergent beam. An additional beam-divider may be used to deflect portion of the emergent beam to the additional detector. Alternatively, (as indicated above) the ‘standard’ remote detector could be moved to intercept the super-deflected beam. Conversely, if a weaker lens than normal is being characterized, the ‘standard’ near detector may be positioned too close to read the emergent beam coordinates with sufficient accuracy and the near detector might be moved further away or a third, more remote detector, with an associated beam-divider may be used for that purpose. Many other arrangements are possible within the scope of the present invention.

As already noted, the method may involve the step of scanning the interrogating beam beyond the edge of the lens so that the edge can be detected and the periphery of the lens can be precisely and automatically determined. This not only allows the entire lens to be mapped but also ensures that the power map is correctly and automatically aligned where the lens does not have a circular periphery or is otherwise asymmetric. Thus, the method can also include the step of determining the edge/boundary, the optical axis, the physical or optical center of the optical system. Similarly, where the lens bears an orientation mark, the method may include the step of detecting and recognizing such a mark. This allows the orientation mark, as well as the peripheral contour to be reproduced with the power map. In the specific case of multi-focal optical systems (such as bifocal ophthalmic lenses), the method may also include the step of detecting the junction/boundary between adjacent optical power zones.

In another variant, the method may include the step of adjusting the angle of the incident beam with respect to the optic axis of the lens being mapped, with concomitant adjustments of the angles and positions of the detectors, as required. This is of particular value with ophthalmic lenses that have a central optic zone surrounded by a peripheral optic zone adapted to adjust the peripheral curvature of field in the manner taught in U.S. Pat. No. 7,025,460 to Smith et al.

From another aspect, the invention includes apparatus, an instrument or a system for use in characterizing an optical system with respect to an optical surface of the system, the apparatus including:
scanner means for moving a narrow incident light beam from spot to spot over the optical surface to generate an emergent beam having an emergent angle for each spot,
detector means adapted to detect and determine the lateral coordinates of the emergent beam at two different optical distances from the optical system, and
processor means adapted to compute the emergence angle for each spot from said lateral coordinates.

The detector means may include separate photodetector arrays located at first and second optical distances from the optical system, each detector array being adapted to output the lateral coordinates of the intersection of an emergent beam with the array. To avoid one array occluding the other, the array(s) closer to the optical system might be moveable to allow the emergent beam to strike a more remote array. Alternatively, the arrays need not be positioned in-line (so that one occludes another) but, instead, the detector means may include beam-divider means that direct a different portion of the emergent beam to each array. In another arrangement, the detector means may include only one photodetector array that can be moved between the first and second optical distances so that the same array is used to determine the lateral coordinates of the emergent beam at each distance or position. As already indicated, the detector means may include more than two separate detector arrays and beam-divider means to direct at least portion of the emergent beam to each.

In one embodiment, the apparatus may be an instrument for mapping the power and/or aberrations of an eye lens (such as a contact lens or spectacle lens). Where the lens is a soft contact lens, it is preferable that it be mounted in a hydrated state—possibly within an aqueous bath—so that it sits upright with its plane horizontal without substantial distortion due to gravity or surface tension, the scanning means may direct the incident beam vertically downwards through the lens, the detector means with its photodetectors and beam-divider (if used) being arranged below the lens. Techniques for mounting and locating soft contact lenses are known in the art and are, for example, employed with Hartmann-Shack instruments.

It will be convenient to employ lasers to generate the incident beams, as is common in the art. The incident beam or beams can be monochromatic with a wavelength selected to suit the purpose of the investigation. More usually, beams with a range of wavelengths that approximate white light in the visible spectrum are appropriate where the lens being characterized is intended for use in association with eyes. However, the use of bi- or poly-chromatic beams is also envisaged to obtain specific spectral characteristics of the device under test.

### DETAILED DESCRIPTION OF EXAMPLES OF THE INVENTION

Having portrayed the nature of the present invention, particular examples will now be described with reference to the accompanying drawings. However, those skilled in the art will appreciate that many variations and modifications can be made to the examples provided without departing from the scope of the invention as outlined above and as claimed below. Also, many other examples are possible within the scope of the invention.

### DESCRIPTION OF THE SEVERAL VIEWS OF THE DRAWINGS

In the accompanying drawings:

FIG. 1 is a diagrammatic perspective of the optical layout of an instrument or system that forms the first example of the invention in which the optical system being characterized is a lens, the incident beam is parallel to the optical axis of the lens and raster-scanned over the surface of the lens and in which a fixed beam-divider is employed.

FIG. 2 is a diagrammatic side elevation of a portion of the instrument of FIG. 1 and comprises a first variant of the instrument of the first example. In this first variant the lens is moved to achieve scanning of the incident beam rather than (or in addition to) moving the incident beam.

FIG. 3 is a diagrammatic side elevation of portion of the instrument of FIG. 1 and comprises a second variant of the instrument of the first example in which the incident beam is pivotally scanned so that it does not remain parallel with the optical axis of the lens.

FIG. 4 is a diagrammatic side elevation of portion of the instrument of FIG. 1 and comprises a third variant of the instrument of the first example in which a pinhole mask is used to effectively scan the incident beam over the surface of the lens.

FIG. 5 is a diagrammatic side elevation of portion of the instrument of FIG. 1 and comprises a fourth variant of the instrument of the first example in which more than two detector planes or positions are employed.

FIG. 6 is a diagrammatic side elevation of portion of the instrument of FIG. 1 and comprises a fifth variant of the instrument of the first example in which an oscillating mirror effectively functions as the beam-divider.

FIG. 7 is a diagrammatic side elevation of portion of the instrument of FIG. 1 in which the lens is tilted so that its non-paraxial power can be mapped or measured at specific locations.

FIG. 8 is a diagrammatic side elevation of the optical layout of an instrument comprising the second example in which one fixed and one moveable array is used, avoiding the need for a beam-divider.

FIG. 9 is a diagrammatic side elevation of the optical layout of an instrument comprising the third example in which a single moveable detector array is used, again avoiding the need for a beam-divider.

FIG. 10 is a diagrammatic side elevation of the optical layout of an instrument comprising the fourth example which is adapted to generate power maps from a reflective optical system, like the human eye, that are useful for the design of corrective modalities.

FIG. 11 is a diagrammatic elevation of the optical layout of an instrument comprising the fifth example in which a single fixed detector array is used, the emergent beam is split and sent through paths of different lengths to the array, and multiplexing of the beam portions is employed.

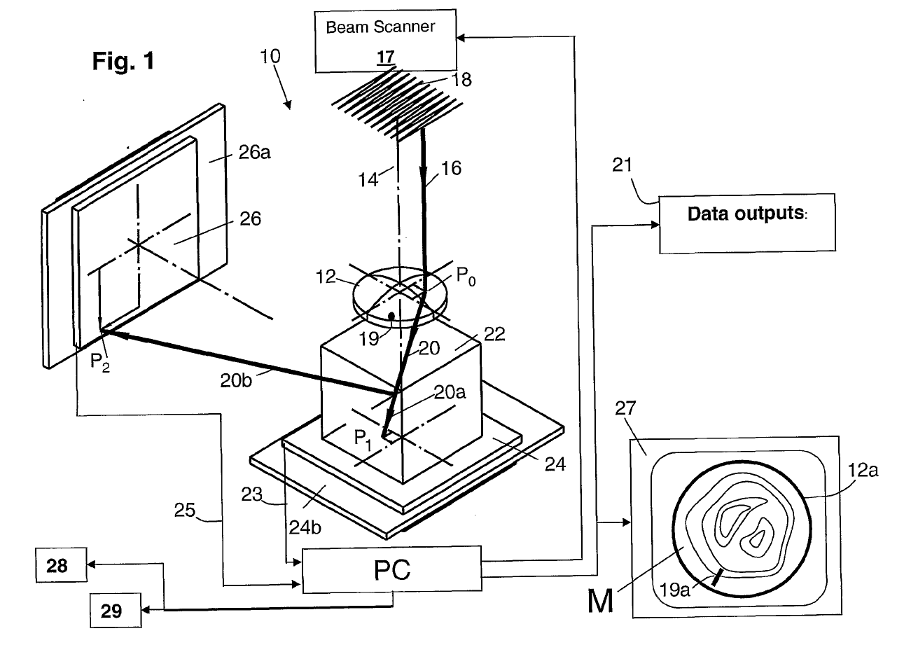

In FIG. 1 the optical layout of system or instrument 10 of the first example is shown in diagrammatic perspective while system components are shown in block diagram form. A simple lens 12 comprises the optical system to be characterized and is shown in position, the optical axis of lens 12 being shown by dot-dash line 14. A narrow incident beam 16 is generated and raster-scanned over the upper surface of lens 12 by a scanner unit 17 so that it remains parallel with axis 14, the raster pattern 18 preferably being larger than lens 12 so that the periphery of the lens can be defined. In this example, lens 12 is asymmetric and has an orientation mark 19 near its periphery.

Scanner unit 17 is connected to and operates under the control of a computer “PC”. Scanner 17 includes a laser light source, an electromechanical scanning device as well as a scanner driver adapted to interface with computer PC, these components not being show as such scanner units are known in the art.

The emergent beam 20 is intercepted by a partially reflective beam-splitter 22 (which serves as a beam-divider) that transmits one portion 20a of emergent beam 20 to a first detector array 24 and reflects a second portion 20b to a second detector array 26, arrays 24 and 26 being located by mounts 24a and 26a that are fixed relative to lens 12 and beam-splitter 22. [Thus, in this example, the detector means comprises beam-splitter 22 and detector arrays 24 and 26.] For convenience, the plane of first detector array 24 is shown orthogonal to axis 14 and optically closer to lens 12 while the plane of array 26 is shown parallel to axis 14 and optically further away from lens 12 than array 24. While this particular arrangement is not essential, it is important that arrays 24 and 26 are positioned at different optical distances from lens 12; one array (say array 24) being at the first optical distance and the other (say array 26) being positioned at the second.

In FIG. 1, scanned incident ray 16 is shown striking lens 12 at a position P₀, the first portion 20a of emergent ray 20 from P₀ is shown striking first photodetector array 24 at point P₁ and the second portion 20b of emergent ray 20 is shown striking second photodetector array 26 at point P₂. Since, in this example, detector arrays 24 and 26 are planar and two dimensional, the coordinates of points P₁ and P₂ can be immediately read-off and input to the I/O interface of computer PC via input lines 23 and 25. Computer PC is configured to compute from these inputs the angle of emergent ray 20 relative to the incident angle of scanned incident ray 16 and, thus, determine the refractive power of lens 12 at spot P₀. This process is repeated for each spot on lens 12 until a data set characterizing the optical properties of lens 12 is generated. This data set can be employed by computer PC to display a contour map M on a monitor 27 of the variation of the refractive power of lens 12 over the surface of the lens. Map M preferably includes a visible mark 19a at the position of orientation mark 19 on lens 12, as well as a representation 12a of the peripheral boundary of lens 12.

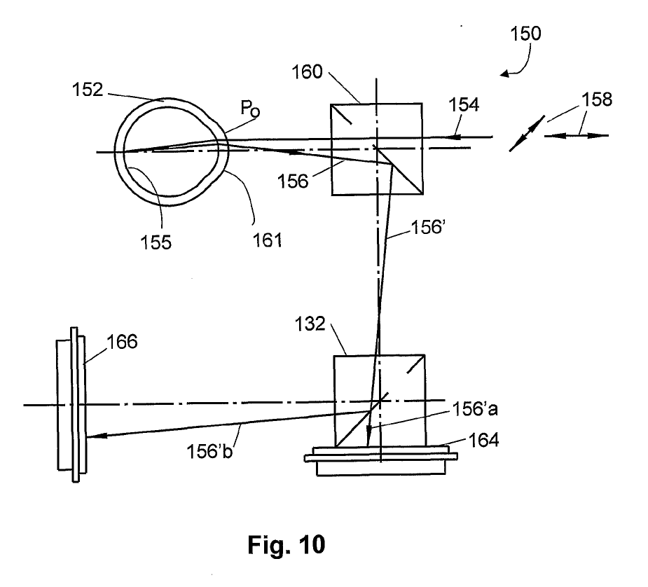

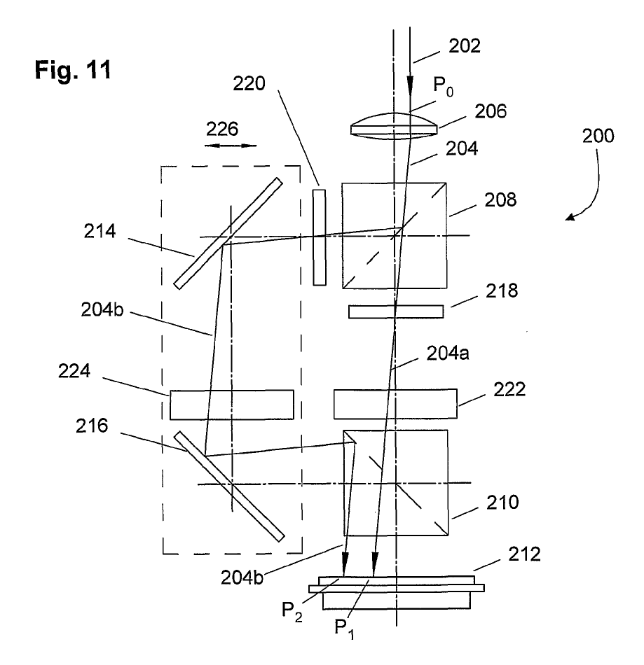

The data set or power map thus generated can be used in many ways known in the art. For example, to compare the designed and measured power profiles to monitor the quality of manufactured lenses, or to compute corrective optics, optical improvements or modifications. This is of considerable importance where an ophthalmic lens is being figured or customized to match and correct the aberrations of a particular human eye. Such a figured surface may be applied to a corrective lens by a machine, indicated at 28. Alternatively, where the optical system is the eye (as described with reference to FIG. 10) the power map of the eye can be used to computer-design and produce a complementary lens or, as is also known in the art, to control a computer-operated laser-surgery machine, indicated at 29, to automatically re-shape the cornea of the eye to correct for refractive error and higher order aberrations.

It will be appreciated by those skilled in the art that the basic optical configuration of instrument 10 of FIG. 1 is such that the deflection of emergent beam 20 relative to incident beam 16 can be computed from the coordinates of points P₁ and P₂ in respective detector arrays 24 and 26 without the need to know the distance of lens 12 from scanner 17. This allows many data outputs to be reliably computer-generated and is a significant advantage over prior art, some of the more important data outputs being indicated in the list included in text box 21 in FIG. 1.

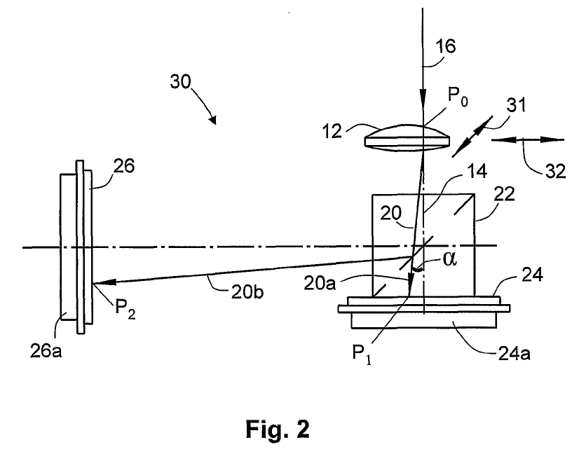

A first variant 30 of the instrument of the first example is diagrammatically illustrated by FIG. 2. The incident beam 16 is not scanned but held steady on optical axis 14 of beam-splitter 22 and lens 12 is moved horizontally in two dimensions—as indicated by arrows 31 and 32—to effectively scan incident beam 16 over lens 12. Thus, emergent angle α of emergent beam 20 can again be computed from the coordinates of incidence of portions 20a and 20b on detector arrays 24 and 26 as described with respect to FIG. 1. As before (and with other variants to be described below) it is desirable—though not essential—for incident beam 16 to be effectively scanned beyond the edge of lens 12 so that the peripheral boundary the lens can be mapped and the orientation of the lens determined by reference to its profile or to orientation marks on its margin.

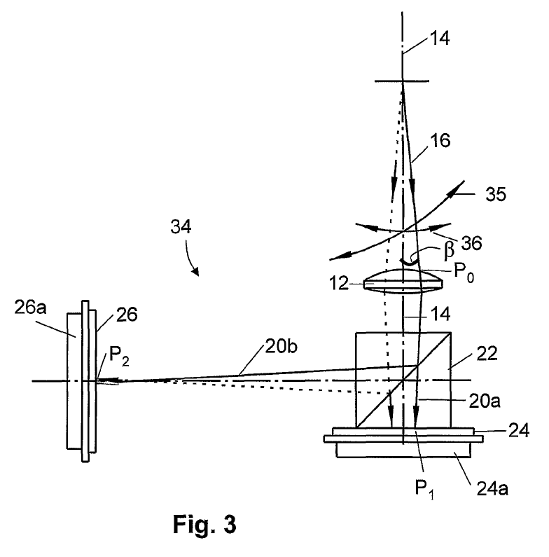

A second variant 34 of the instrument of FIG. 1 is shown in FIG. 3 in which the incident beam 16 is pivotally or conically scanned over the surface of lens 12, as indicated by arrows 35 and 36. While scanned incident beam 16 will not remain parallel with optical axis 14 of lens 12, its angle of incidence β relative to axis 14 at any spot P₀ will be known from the scanner driver of scanner unit 17 [FIG. 1] and/or scanner control software/firmware within processor PC [FIG. 1]. Again, the emergent angle α [FIG. 2] of emergent beam 20 can be computed from the coordinates of points P₁ and P₂ and used, together with the angle β of incident beam 16 to determine the refractive power of each spot P₀ on lens 12.

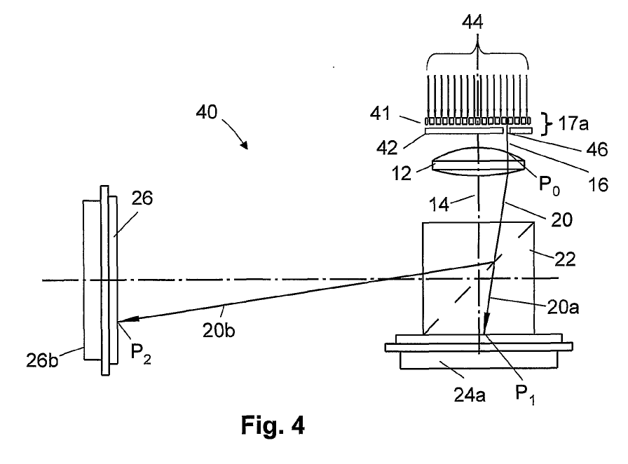

In the third variant 40 of the first example shown in FIG. 4, the scanner unit, indicated at 17a, is formed by an aperture plate 41 and a pinhole mask 42 that is controlled by computer PC [FIG. 1]. Aperture plate 41 is illuminated by a broad collimated light beam 44 and passes light through a large number of small apertures that form a two-dimensional array of apertures in plate 41. Mask 42, which can comprise a selectively transparent LCD device known in the art, is then controlled to permit light from a selected aperture in plate 41 to pass through mask 42 at an aligned spot—indicated at 46—which has been made transparent. Indeed, with suitable LCD mask devices 42, separate aperture plates 41 may not be needed. In any event, mask 42 serves to generate a scanned incident beam 16 that is parallel to optical axis 14 and, therefore, has a zero incident angle β [FIG. 3] as in the instrument of FIG. 1. As in FIGS. 1 and 2, the detector means here comprises two detector arrays and one beam-splitter. On the other hand, scanner means 17a comprises aperture plate 41 and mask 42, along with a light source and mask interface with computer PC which are not shown.

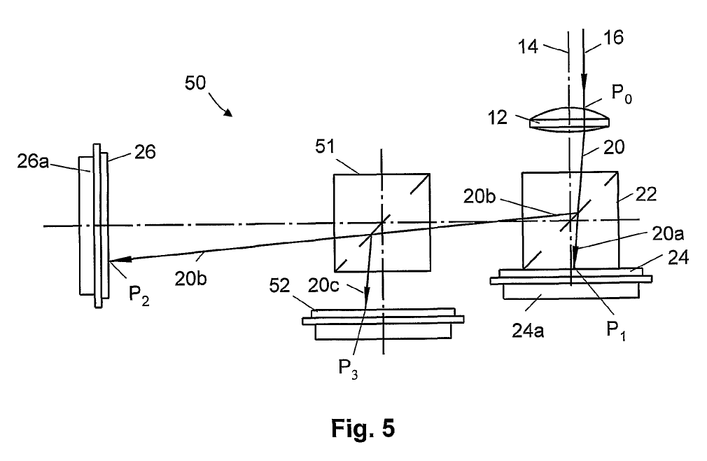

FIG. 5 illustrates a fourth variant 50 of the instrument of FIG. 1 that allows more than two detector arrays to be used so that the angle of emergent beam 20 can be computed more accurately and/or so as to extend the measurement range. In this variant, first and second emergent beam portions 20a and 20b are generated by beam-splitter 22 and directed to first and second detector arrays 24 and 26 as in FIG. 1, but beam portion 20b is passed through a second beam-splitter 51 that generates a third beam portion 20c which is directed to a third detector array 52 where beam portion 20c strikes at point P₃. In the configuration shown, array 26 is optically further away from lens 12 than array 52 which is, in turn, further away than array 24. The outputs of all three arrays 24, 26 and 52 are sent to computer PC to provide the intersection coordinates of points P₁, P₂, and P₃, if present. The arrangement of the detector arrays is such that emergent beam portions 20a and 20c will always intersect respective arrays 24 and 51 over the full range of measurable power, but beam portion 20b will only strike array 26 over a lower part of the range of measureable power. Over this lower power range the outputs of detector arrays 24 and 26 can be used to allow more accurate power computation. Over the remaining higher power range (where beam portion 20b does not strike detector 26), the outputs of arrays 24 and 51 will be used. Use of more than two detector arrays in this way can therefore allow a wider range of power measurement than in a two-detector arrangement (as in FIG. 1) and/or to permit higher precision measurements over a lower-power range. It will be noted that, in this variant, the detector means comprises three detector arrays and two beam-splitters.

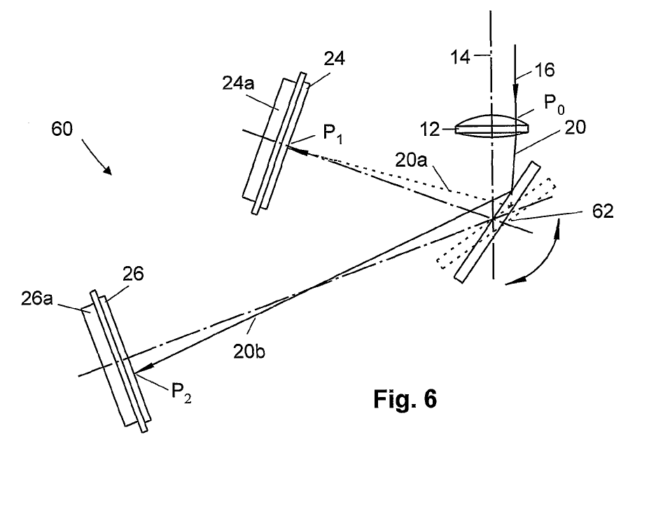

The fifth variant 60 of the first example shown in FIG. 6 employs an oscillating or rotating mirror 62 as a beam-divider (instead of partially-reflective beam-splitter 22 of FIG. 1). Mirror 62 switches emergent beam 20 between detector arrays 24 and 26 in quick succession, effectively generating the emergent beam portions 20a and 20b (and 20c, if desired) as before. Of course, as is well known in the art, mirror 56 can be replaced by a prism, lens or multi-faceted reflector to effect the scanning/beam-splitting function. A single beam-divider of this type has the advantage that it can readily direct emergent beam 20 to two or more detector arrays with undiminished intensity but the disadvantage of relative slowness when compared with fixed partially reflective beam-splitters. In variant 60, the detector means comprises moving mirror 62 as well as detector arrays 24 and 26.

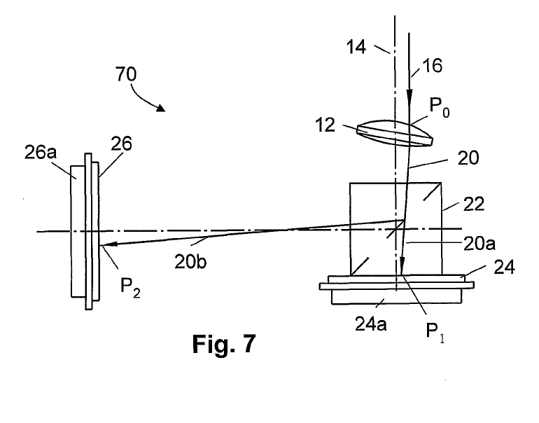

FIG. 7 shows a sixth variant 70 of the first example in which lens 12 is tilted relative to axis 14 so that non-paraxial power can be mapped over portions—or all—of its surface, or measured at specific locations. Lens 12 can be mounted in a goniometer-like holder (not shown) so that it can be angled precisely relative to axis 14 in many different planes.

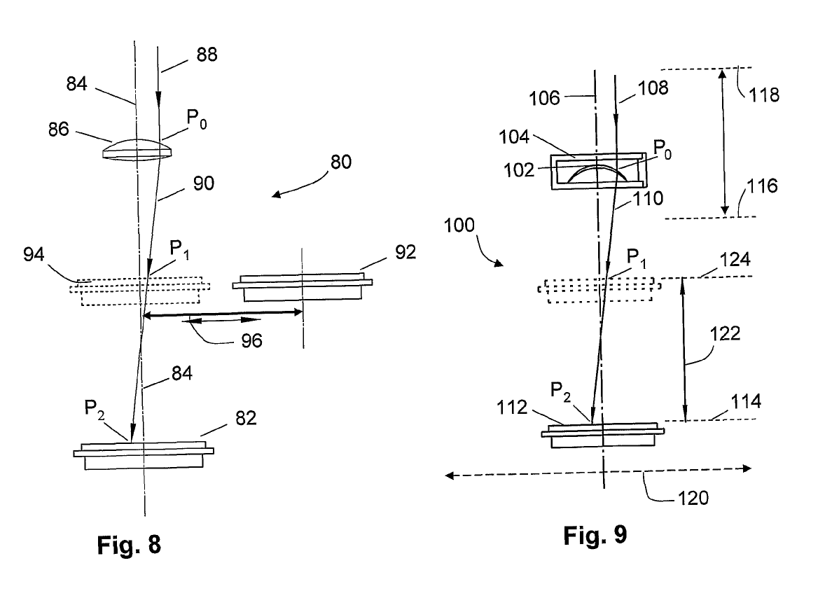

FIG. 8 illustrates the second example which avoids the need for any beam-divider. The instrument 80 of this example employs a fixed photodetector array 82 positioned on optical axis 84 of the instrument opposite the lens 86 which serves as the optical system to be characterized. As before the incident beam 88 is scanned over the surface of lens 86 and is shown impinging on spot P₀ on the surface, generating an emergent beam 90 which strikes array 82 at the coordinates of point P₂. To obtain the second set of coordinates at a closer point to lens 86, a second detector array 92 is slid laterally until it is aligned with axis 88 where it is shown in broken lines 94. With array 92 so positioned, array 94 occludes the more distant array 82 and emergent beam 90 now impinges at point P₁ on array 92 providing the set of coordinates of emergent beam at this location (optical distance from lens 86). The coordinates so obtained can then be employed to characterize the optical system (86) as described with respect to the first example. Of course, After both sets of coordinates have been recorded for spot P₀ on lens 86, moveable array 92 is returned to its original position (where array 92 is shown in solid lines), incident beam is moved to a new spot on lens 86 and the process is repeated so as to record both sets of coordinates for the new spot. The reciprocating movement of array 92 in this process is indicated by arrow 96.

FIG. 9 is a diagrammatic side elevation of the optical layout of an instrument 100 comprising the third example of the invention for use in characterizing a hydrated soft contact lens 102 located in and supported by a wet cell 104. In this example, the optical axis of lens 102 is indicated at 106, scanned incident beam at 108, and the emergent beam at 110. Incident beam 108 is again shown as striking lens 102 at spot P₀. In this example, not only is the need for a beam-divider avoided but only a single photodetector array is required.

In instrument 100 the single detector array 112 is fixed at a location indicated by broken line 114, array 112 being drawn in solid lines at this position. Wet cell 104, together with soft contact lens 102 that serves as the optical system under investigation, is moveable up and down along optical axis 106 between two positions, indicated at 116 and 118, position 116 being a shorter optical distance from detector array 112 than position 118. If desired, cell 104 can be moved to one or more intermediate positions for scanning by incident beam 108 to exploit the advantages mentioned in connection with instrument 50 of FIG. 5 where more than two optical distances between lens and detector array were employed. Indeed, further advantages in terms of measurement range can be obtained by mounting array 112 for sideways movement, as indicated by broken-line arrow 120. This enables instrument 100 to record the characteristics of lenses that are more highly refracting than those that can be measured with array 120 in fixed position.

In a second configuration of instrument 100, array 112 can be moved up and down along axis 106 so as to be closer to or further away (optically) from lens 102 in wet cell 104, as indicated by arrow 122. For simplicity, it is assumed that only two positions, indicated at 114 and 124 are required, though one or more intermediate positions can be readily employed with the advantages mentioned in connection with instrument 50 of FIG. 5. Array 112 is shown in full lines at the most distant position 114 and in broken lines at the closest position 124 where it is indicated at 112a. Again, if desired, array 112 might be moveable laterally at position 114 (and/or at position 124) to allow a wider range of lenses 102 to be characterized (as indicated by dashed-line arrow 120 and as described above). Indeed, an even greater measurement range could be accommodated if both lens 102 and cell 104 were also moveable as described above.

While this example of an instrument for characterizing an optical system such as a contact lens eliminates the need for a beam-divider, the need to accurately move the detector array(s) and/or the optical system in one or two dimensions will add cost and complication.

The third example of a measurement instrument or system formed in accordance with the present invention is shown in FIG. 9. In this example, instrument 150 enables reflective (including back-scattering) optical systems to be mapped or otherwise characterized, whether they are simple mirrors, figured reflective surfaces, or complex optical systems such as the human eye. As shown in FIG. 9, the optical characteristics of a human eye 152 having an optical axis 153 can be assessed by scanning an incident beam 154 into eye 152 by making use of reflection or back-scattering from the retina 155 to generate a return or emergent beam 156, the scanning of incident beam 154 being indicated by arrows 158, a scanner unit not being otherwise shown in FIG. 9. Scanned incident beam 154 is passed directly through a first beam-splitter 160, through the cornea 161 of eye 152, at position P₀ on cornea 161, and onto retina 155. Returned emergent beam 156 passes back into beam-splitter 160 and is reflected, as indicated at 156′, into a second beam-splitter 162 which (as in beam-splitter 22 of FIG. 1) directs a first portion 156′a of emergent beam 156′ to a first or ‘close’ detector array 164 and a second portion 156′b of the emergent beam to a second or ‘distant’ detector array 166. As described for the first example, arrays 164 and 166 enable the lateral coordinates emergent ray 156 to be determined at two different optical distances, from which sets of coordinates the relative angle between incident and emergent rays 154 and 156 can be determined and used as the basis for generating the sorts of data outputs indicated in FIG. 1 that can be mapped onto the surface of cornea 161.

It is convenient to map the optical characteristics of eye 152 onto the surface of cornea 161 rather than retina 125 because the cornea is readily visualized and, in procedures involving the reshaping of the cornea, the contour or profile of the cornea surface prior to the reshaping procedure is determined with considerable accuracy. The combination of mapping the power of the eye onto the cornea with determination of the profile of the cornea provides near-complete information necessary for cornea modification as well as the provision of tailored corrective ophthalmic lenses. However, the optical characteristics of the eye could be mapped onto the surface of the retina, or any other interface that can be visualized within the eye, if desired.

The fourth exemplary embodiment of the invention is instrument 200 shown in FIG. 10 which employs a single fixed detector array to output the lateral coordinates of the emergent beam at two optical distances from the optical system. Referring more specifically to FIG. 10, the optical axis of the system is shown at 201, incident beam at 202, emergent beam at 204, the lens (the optical system being characterized) at 206 and the single detector array at 207. As in the first example [FIG. 1], incident beam 202 is scanned over the surface of lens 206 and is shown at the instant when it impinges on spot P₀. While only one detector array is used, the detector means—indicated by broken-line box 209—is quite complex and will be described below.

Emergent beam 204 is split by beam-splitter 208 into two portions. A first portion 204a travels direct to array 207 over a short optical distance via a second beam-splitter 210 and a second portion 204b travels indirectly over a longer optical distance to array 207, also via second beam-splitter 210. The greater optical distance for beam portion 204b is achieved by reflecting beam 204b laterally in splitter 208 to a first mirror 214, from which it is reflected to a second mirror 216 that reflects it back to second beam-splitter 210 from which it is finally reflected to array 207 along with the first beam portion 204a.

Confusion between beam portions 204a and 204b at array 207 can be avoided in a number of ways, the most convenient being discrimination by optical properties, time multiplexing or pulse-encoding. Discrimination by optical properties can be implemented using a first optical filter 218 in the path of beam portion 204a and a second filter 220 in the path of beam portion 204b. Filters 218 and 220 can be polarizing, chromatic or intensity; the choice depending upon the characteristics of detector array 207. Thus, if array 207 is color sensitive, filter 218 might be a chromatic red filter and filter 220 might be a chromatic green filter so that beam portions 204a and 204b can be readily distinguished by array 207. Time multiplexing can be implemented by introducing a first mechanical or opto-electronic chopper 222 into the path of beam portion 204a and a similar chopper 224 into the path of beam portion 204b. Choppers 222 and 224 are operated so that beams 204a and 204b are alternately blocked so that they are presented alternately to array 207, a form of time-division multiplexing. This requires the computer system (not shown here) to keep track of which beam is present in each time-slot, a matter that those skilled in the art will be able to implement. Pulse encoding can be implemented by using only one of choppers 222 or 224, say chopper 222 in the path of beam 204a. This chopper is operated significantly faster than the scanning of incident beam 202 from spot to spot on lens 206 so that the signal from beam 204a appears as AC while that from beam 204b appears as DC during the time interval that incident beam 202 remains on spot P₀. These two signals will then be readily distinguished by well known electronic filtering techniques.

Another optional refinement of instrument 200 allows the path length of beam portion 204b to be lengthened as desired so as to greatly increase the sensitivity of the instrument where the optical system being characterized either has unusually low refractive power or aberrations. In this option, the sub-assembly indicated by box 226 comprising mirrors 214 and 216 can be moved laterally towards and away from beam-splitters 208 and 210 to alter the optical distance between the optical system [lens 206 in this case] being characterized and detector array 207 for beam portion 204b only. This variation of instrument 200 can be extended to include more than two optical path lengths. More beam-dividers can be added to generate several optical path lengths, before recombining them to be detected by a single detector. The use of other variations described in the previous examples—such as the substitution of a moving-mirror beam-divider for beam-splitter 208 and/or 210—is also envisaged.

1. A method of characterizing an optical system comprising:
directing an incident light beam onto successive spots on an optical surface of the optical system to generate an emergent beam for each spot,
determining the lateral location of each emergent beam at a first optical distance from the optical system,
determining the lateral location of each emergent beam at a second optical distance from the optical system, and
deriving the power of the optical system at each respective spot on the optical surface by employing the determined lateral locations of the emergent beam.

2. A method according to claim 1 including the steps of:
employing said determined lateral locations of the emergent beam to calculate the angle of the emergent beam at each respective spot, and
deriving the power of the optical system at each respective spot from said calculated angle of the emergent beam at said spot.

3. A method according to claim 1, wherein:
the lateral location of each emergent beam at said first optical distance is determined by determining the lateral spatial coordinates of each emergent beam at said first optical distance, and
the lateral location of each emergent beam at said second optical distance is determined by determining the lateral spatial coordinates of each emergent beam at said second optical, distance.

4. A method for mapping the optical power of an optical system over an optical surface of the system, the method comprising:
causing an incident light beam to strike each successive spot on the surface at a known incident angle so as to generate an emergent light beam for each successive spot,
for each successive spot, determining first lateral spatial coordinates of said emergent beam at a first optical distance from the optical system,
for each successive spot, determining second lateral spatial coordinates of said emergent beam at a second optical distance from the optical system, said second optical distance being greater than said first optical distance, and
for each successive spot, deriving the optical power of the optical system at said spot by (i) computing the angle of the emergent beam from said first and second coordinates and (ii) comparing said computed angle of the emergent beam with the incident angle of the respective incident beam at said spot.

5. A method according to claim 4, including the step of:
causing said incident light beam to strike each successive spot on said optical surface by moving the incident beam.

6. A method according to claim 4, including the step of:
causing said incident light beam to strike each successive spot on said optical surface by moving said optical surface.

7. A method according to claim 4, including the step of:
successively generating a separate incident beam for each spot on said surface.

8. A method according to claim 4, wherein:
said optical system has an optical axis that passes through said optical surface, and
said incident beam is and remains parallel with said optical axis at each spot on said surface.

9. A method according to claim 4, including the steps of:
directing said emergent beam for each spot through said first optical distance to a first photodetector array and using said first array to determine said first lateral spatial coordinates, and
directing said emergent beam for each spot through said second optical distance to a second photodetector array and using said second array to determine said second lateral spatial coordinates.

10. A method according to claim 4, including the steps of:
moving a photodetector array to intercept the emergent beam at said first optical distance to determine said first lateral spatial coordinates, or
moving said photodetector array to intercept the emergent beam at said second optical distance to determine said second lateral spatial coordinates.

11. A method according to claim 4, including the steps of:
directing the emergent beam from each spot to a photodetector array,
changing the optical distance between the optical system and the said photodetector array to equal said first optical distance to determine said first lateral spatial coordinates, and
changing the optical distance between the optical system and the said photodetector array to equal said second optical distance to determine said second lateral spatial coordinates.

12. A method according to claim 4, including the steps of:
dividing said emergent beam into a first portion and a second portion,
directing said first portion via said first optical distance to allow determination of said first spatial lateral coordinates of the emergent beam, and
directing said second portion via said second optical distance to allow determination of said second spatial lateral coordinates of the emergent beam.

13. A method according to claim 12, including the steps of:
additionally dividing said emergent beam into a third portion,
directing said third portion via a third optical distance,
determining the lateral spatial coordinates of the third portion of the emergent beam at a third optical distance from the optical system, and
using at least two of said first, second and third lateral spatial coordinates to determine the emergent angle of the emergent beam at said spot.

14. A method according to claim 12, wherein said emergent beam is divided by a method selected from the group consisting of:
using a partially reflective beam-splitter,
using a moving reflector to switch the emergent beam,
differentially modulating and detecting at least one of said beam portions, or
differentially changing the optical characteristics of at least one of said beam portions and differentially detecting said changed beam portion.

15. A method according to claim 12, including the steps of;
directing at least two of said beam portions to a common photodetector array, and
employing said common array to differentially determine the respective spatial coordinates of each of said beam portions at said array.

16. A method according to claim 1, wherein the optical system is an ophthalmic lens, said optical surface comprises the anterior or the posterior surface of the lens and wherein the lens has a peripheral boundary, the method including the steps of:
directing the incident beam beyond the peripheral boundary of the lens,
detecting the boundary of the lens so as to enable orientation of the spots on the optical surface with respect to said detected boundary.

17. A method according to claim 1, wherein:
the optical system is an eye having a cornea and a retina,
said optical surface comprises the cornea surface, and
said emergent beam is generated by reflection or scattering of the incident beam from the retina back through the cornea.

18. An instrument for use in characterizing an optical system with respect to an optical surface of the system, comprising:
a scanner for moving a narrow incident light beam from spot to spot over the optical surface to generate an emergent beam having an emergent angle for each spot,
a detector to detect and determine the lateral spatial coordinates of the emergent beam for each spot at least two different optical distances from the optical system, and
a processor to compute the emergent angle of the emergent beam for each spot from said determined lateral coordinates.

19. An instrument or apparatus adapted to indicate the variation of optical power of an optical system over an optical surface of the system, the instrument or apparatus comprising:
a scanner to sequentially scan a narrow light beam from spot to spot on the optical surface at a known incident angle at each spot and to thereby generate an emergent beam from each spot having an emergent angle,
detector to generate a first output indicative of first lateral coordinates of the emergent beam at a first optical distance from the optical surface, and adapted to generate a second output indicative of second lateral coordinates of the emergent beam at a second optical distance from the optical surface, and
processor to receive said first and second outputs and to determine the emergent angle of the emergent beam at each spot on the optical surface from said first and second outputs, compute the difference between the emergent angle and the incident angle at each spot and to be capable of indicating the variation of optical power over said optical surface.

20. An instrument according to claim 19, wherein:
said detector also generates a third output indicative of third coordinates of the emergent beam at a third optical distance from the optical surface, each of said optical distances differing from one another, and
said processor also receives said third output and employs said third output, in addition to at least one of said first and second outputs, to determine the emergent angle of the emergent beam.

21. An instrument according to claim 18, wherein the detector includes:
a two-dimensional photodetector array adapted to output the lateral spatial coordinates of a light beam incident thereon, and
at least one beam divider located between the optical system and said array adapted to intercept the emergent beam from each spot and to direct a first portion thereof to said array through said first optical distance and to direct a second portion thereof to said array through said second optical distance.

22. An instrument according to claim 18, wherein the detector includes:
a first two-dimensional photodetector array adapted to output the spatial coordinates of a light beam incident thereon, said first array being arranged at said first optical distance from the optical surface,
a second two-dimensional photodetector array adapted to output the spatial coordinates of a light beam incident thereon, said second array being arranged at said second optical distance from the optical surface.

23. An instrument according to claim 22, including:
at least one beam-divider located between the optical system and each of said detector arrays adapted to intercept the emergent beam from each spot and to direct a first portion thereof to said first array and a second portion thereof to said second array.

24. An instrument according to claim 18, wherein said detector means comprises:
a two-dimensional photodetector array adapted to output the lateral spatial coordinates of a light beam incident thereon, said array being moveable to intercept at least a portion of the emergent beam at said first optical distance from the optical surface and/or being moveable to intercept at least a portion of the emergent beam at said second optical distance from the optical surface.

25. An instrument according to claim 18, wherein:
said optical system comprises an ophthalmic lens having a principal optical surface, an optical axis and a peripheral boundary,
said optical surface comprises at least portion of said principal surface,
said scanner is adapted to scan said incident beam over the optical surface while maintaining said beam parallel with said optical axis,
the scanner is adapted to scan said beam beyond said peripheral boundary of the lens, and
said processor means is adapted to identify the peripheral boundary of the lens and to indicate the variation of optical power over said optical surface within said boundary.

26. An instrument according to claim 18, wherein:
said optical system comprises an ophthalmic lens having a principal optical surface, an optical axis and a peripheral boundary,
said scanner is adapted to direct said incident beam onto said optical face and to move the lens in a controlled manner to effectively scan the incident beam from spot to spot on the optical surface.

27. An instrument according to claim 18, wherein:
said optical system comprises an eye having an cornea and a retina, said optical surface comprising at least portion of the cornea,
the scanner is arranged to direct the incident beam into the eye through the cornea so that the emergent beam is reflected or back-scattered from the retina back through the cornea, and
the detector includes a beam-divider positioned so that the incident beam passes therethrough along an incident path and the beam-divider is adapted to intercept and deflect the emergent beam from said incident path.

28. A method of characterising an optical system at a position on an optical surface of the optical system, the method comprising:
directing an incident light beam at the position on the optical surface;
detecting the lateral location of an emergent light beam from the optical system resulting from the incident light beam at a first optical distance and at a second optical distance, different from the first optical distance;
determining a measure of the angle between the incident light beam and the emergent light beam from the difference between the first optical distance and the second optical distance and from the detected lateral locations at the first optical distance and the second optical distance;
wherein determining a measure of the angle between the incident light beam and the emergent light beam is made independently of the spatial location of the optical system.

29. The method of claim 28, wherein detecting the lateral location of an emergent light beam at the first optical distance and the second optical distance comprises:
dividing the emergent light beam into a first portion and a second portion, and
detecting the lateral location of the first portion at the first optical distance and detecting the lateral location of the second portion at the second optical distance.

30. The method of claim 28, wherein detecting the lateral location of an emergent light beam at the first optical distance and the second optical distance comprises moving one or both of the optical system and a detector used for detecting the lateral location of the emergent light beam.

31. The method of claim 28, wherein the optical surface has a curved profile and the method further includes repeating the processes of directing, detecting and determining at least at one other position on the optical surface, which has a different distance from the source of the incident light beam.

32. Apparatus for characterising an optical system, the apparatus comprising:
means for generating a beam of light that travels along an axis to intersect the optical system;
means for detecting the lateral location of an emergent light beam from the optical system resulting from the generated beam of light both at a first optical distance and at a second optical distance, different from the first optical distance; and
means for determining a measure of the angle between the incident light beam and the emergent light beam from the difference between the first optical distance and the second optical distance and from the detected lateral locations at the first optical distance and the second optical distance;
wherein the means for determining a measure of the angle between the incident light beam and the emergent light beam makes the determination independently of the spatial location of the optical system.

33. The apparatus of claim 32, comprising means for dividing the emergent light beam into a first portion and a second portion, wherein detecting the lateral location of an emergent light beam from the optical system comprises means for detecting the lateral location of the first portion and separate means for detecting the lateral location of the second portion.

34. The apparatus of claim 33, wherein the means for dividing the emergent light beam comprises a beam splitter.

35. The apparatus of claim 33, wherein the means for dividing the emergent light beam comprises a movable reflector.

36. The apparatus of claim 32, comprising means for moving at least one of the optical system and the means for detecting the lateral location of an emergent beam from the optical system, said means for moving creating the first and second optical distances.
